(19) United States
(12) Patent Application Publication
(10) Pub. No.: US 2016/0135755 A1
(43) Pub. Date: May 19, 2016

(54) Remote home healthcare system

(71) Applicant: ZTE Corporation, Shenzhen, Guangdong (CN)

(72) Inventors: Ping Lu, Shenzhen (CN); Shuo Deng, Shenzhen (CN); Mengqian Lou, Shenzhen (CN); Yi Xie, Shenzhen (CN); Zhixin Sun, Shenzhen (CN)

(21) Appl. No.: 14/437,293

(22) PCT Filed: Aug. 19, 2013
(86) PCT No.: PCT/CN2013/081738

§ 371 Date: Jul. 1, 2015

(30) Foreign Application Priority Data
Aug. 19, 2013 (CN) 201210409115.7

(51) Int. Cl. A61B 5/00; A61B 5/0205

(52) U.S. Cl. CPC A61B 5/7267; A61B 5/7282; A61B 5/7203; A61B 5/7246; A61B 5/746; A61B 5/02055; A61B 5/4815; A61B 5/7225; A61B 5/7275; A61B 5/6889; A61B 5/7221; A61B 2560/0475; A61B 2505/07; A61B 5/1117

(57) ABSTRACT

A remote home health care system is described. The system includes: a fusion sorting subsystem configured to receive physical sign data parameters collected by a sensor in real time, perform fusion sorting processing on the physical sign data parameters, pre-diagnose a physical condition of a user in real time according to physiological data and a physiological model in a physiological model library and feed back the pre-diagnosed physical condition; a resource optimization subsystem configured to optimize the physiological data in a physiological database periodically, generate a personalized physiological model for the user according to historical physiological data in the physiological database, store the generated physiological model in the physiological model library, and update the physiological model in the physiological model library according to the latest physiological data in the physiological database; and a comprehensive evaluation subsystem configured to predict a changing trend and a dynamic change range of the physical signs of the user according to the physiological data in the physiological database and the physiological model in the physiological model library and evaluate a health condition of the user according to the physiological data and the result of the prediction.

### TECHNICAL FIELD

The disclosure relates to the computer field, and more particularly to a remote home health care system.

### BACKGROUND

In related art, a home medical monitoring system can receive physical sign information collected by various biosensors and transmit the received physical sign information to a remote monitoring center through a network so that each physical index of a person under observation can be continuously observed for a long time, thus realizing the purpose of monitoring the health condition of the person under observation and giving an alarm when there is an abnormality. Remote expert consultation and health evaluation refers to that health consultants read the heath records of the person, evaluate the current health condition of the user and offer a corresponding health care guidance.

At present, there are the following specific defects in related art:

1: lack of an intelligent diagnostic technology: after existing remote home medical monitoring system transmits a great amount of data to a remote monitoring center, the data is mainly monitored manually, and a heath consultation is also mainly conducted manually, which not only increases the workload of a doctor but also makes it difficult to improve the efficiency of a system.

2: non-personalized: existing home medical monitoring systems all give a diagnostic alarm in a threshold alarming manner and are therefore non-personalized; the people under observation are physically different, thus requiring a personalized intelligent auxiliary diagnostic technology.

3: causing error historical data and the loss of historical data: lack of necessary maintenance and optimal management, the physiological data collected and recorded for a user and the health records of the user are generally damaged and lost, thus, certain recovery and remedy approaches are needed.

4: high false alarm rate: a biosensor occasionally losses efficacy in precision and accuracy, besides, a simple threshold alarming method is likely to lead to an error health condition evaluation or an omitted health condition evaluation. How to obtain more effective and consistent data by getting rid of error information and contradictory information from a great amount of data to improve the accuracy and the credibility of information becomes an important problem needing solving urgently. High false alarm rate not only causes problems to the normal life of the user but also results in distrust on an alarm signal; as a consequence, the treatment of a real disease may be delayed.

In view of the problems above, it is urgently needed to provide an intelligent personalized health detection and evaluation solution to a remote home medical monitoring system.

### SUMMARY

A remote home health care system is provided to address the problem that existing remote home medical health care systems are commonly high in false alarm rate and lack of an intelligent personalized health diagnostic technique and commonly cause error historical data and the loss of historical data.

A remote home health care system is provided which includes: a fusion sorting subsystem configured to receive physical sign data parameters collected by a sensor in real time, perform fusion sorting processing on the physical sign data parameters and pre-diagnose a physical condition of a user in real time according to the physical sign data parameters and a physiological model in a physiological model library while discovering and filtering error data in the physical sign data parameters, and store data resulting from the fusion sorting processing in a physiological database as physiological data;

a resource optimization subsystem configured to recover and optimize the physiological data in the physiological database, generate a personalized physiological model for the user according to historical physiological data in the physiological database, store the generated physiological model in the physiological model library, and update the physiological model in the physiological model library according to the latest physiological data in the physiological database;

a comprehensive evaluation subsystem configured to predict a changing trend and a dynamic change range of the physical signs of the user according to the physiological data in the physiological database and the physiological model in the physiological model library, and evaluate a health condition of the user according to the physiological data and the changing trend and the dynamic change range of the physical signs;

a physiological database configured to store the physiological data of the user; and

a physiological model library configured to store the physiological model of the user.

Preferably, the physiological data in the physiological database may include: physical sign data, electronic medical record and health record.

Preferably, the fusion sorting subsystem may be further configured to delete the error data in the physical sign data parameters through the fusion sorting processing before storing the physical sign data parameters in the physiological database.

Preferably, the fusion sorting subsystem may include:

a motion state detection module configured to detect whether the user falls or is in motion according to the physiological data collected by the sensor in real time, give a fall alarm or an abnormal body position alarm and send the fall alarm or the abnormal body position alarm to an alarming module after detecting that the user falls, or send motion information to a health detection module after detecting that the user is in motion;

a health detection module configured to perform data fusion correlation processing and historical data correlation processing according to the acquired physiological data and motion information, diagnose a disease and discover a physiological data error according to corresponding physiological data and a corresponding physiological model, output a corresponding disease pre-diagnosis result, give a disease alarm if the disease pre-diagnosis result is abnormal, send the disease pre-diagnosis result and the disease alarm to the alarming module, and send a physiological data error signal to an error location module;

an error location module configured to receive the physiological data error signal sent from the health detection module, position a faulted sensor and give a sensor error alarm to prompt the user to check a corresponding sensor; and

an alarming module configured to conduct a comprehensive calculation according to the fall or abnormal position body alarm sent from the motion state detection module and the disease pre-diagnosis result and the disease alarm sent from the health detection module, output final alarm information, automatically give an alarm to a medical institution and/or a family member of the user after determining that the user is in danger according to the final alarm information and send the currently abnormal physiological data of the user.

Preferably, the health detection module may be configured to:

perform the data fusion correlation processing on the various physiological data acquired;

perform the historical data correlation processing according to the various physiological data collected by the sensor in real time and the historical physiological data stored in the physiological database using the following formula 1:

PD(tₙ)=CP(tₙ)−NP(tₙ) Formula 1

where tₙ is any time of a day, PD is a physical sign difference, CP is a current detection value of a certain physical sign, and NP is a physical sign reference value.

Preferably, the health detection module may include:

a fever detection module configured to carry out the historical data correlation processing according to the various physiological data collected by the sensor in real time and the historical physiological data stored in the physiological database, determine whether or not the user has a fever according to the motion information and a corresponding physiological model, discover a physiological data error, output a fever pre-diagnosis result and give a fever alarm if the fever pre-diagnosis result is abnormal, wherein the acquired physiological data includes: a body temperature parameter and a heart rate parameter;

a cold detection module configured to carry out the historical data correlation processing according to the various physiological data collected by the sensor in real time and the historical physiological data stored in the physiological database, determine whether or not the user has a cold according to the motion information, corresponding physiological data and a corresponding physiological model, discover a physiological data error, output a cold pre-diagnosis result and give a fever alarm if the cold pre-diagnosis result is abnormal, wherein the acquired physiological data includes: a body temperature parameter, a heart rate parameter and a blood oxygen parameter;

a heart blood pressure detection module configured to carry out the data fusion correlation processing according to the heart rate parameter, a systolic pressure parameter and a diastolic pressure parameter contained in the various physiological data collected by the sensor in real time, perform the historical data correlation processing on original input parameter, the parameters resulting from the fusion processing, that is, an ambulatory pulse pressure, a mean arterial pressure and an ambulatory rate-pressure product, and the historical physiological data stored in the physiological database, determine whether or not the user suffers a cardiac disorder and/or a blood pressure disorder according to the motion information, corresponding physiological data and a corresponding physiological model, discover a physiological data error, output a heart blood pressure pre-diagnosis result and give a heat blood pressure alarm if the heart blood pressure pre-diagnosis result is abnormal, wherein the acquired physiological data includes: a heart rate parameter, the systolic pressure parameter and the diastolic pressure parameter; and

a sleep quality detection module configured to carry out the data fusion correlation processing according to the heart rate parameter, the systolic pressure parameter and the diastolic pressure parameter contained in the various physiological data collected by the sensor in real time, perform the historical data correlation processing on original input parameters, the parameters resulting from the fusion processing, that is, the ambulatory pulse pressure, the mean arterial pressure and the ambulatory rate-pressure product, and the historical physiological data stored in the physiological database, determine whether or not the sleep quality of the user is abnormal according to the motion information, corresponding physiological data and the corresponding physiological model, discover a physiological data error, output a sleep quality pre-diagnosis result and give a sleep quality alarm if the sleep quality pre-diagnosis result is abnormal, wherein the acquired physiological data includes: the heart rate parameter, the systolic pressure parameter, the diastolic pressure parameter and the blood oxygen parameter.

Preferably, the error location module may be configured to: start a re-transmission mechanism for a faulted sensor after locating the faulted sensor, start a sensor error alarm to prompt the user to check a corresponding sensor if the error still exists after re-transmission is carried out more than a predetermined threshold times.

Preferably, the error location module may be configured to acquire a location output signal according to the following formula 2:

Le=He*2³+Ce*2²+Be*2¹+Se*2⁰ Formula 2

where Le is a location output signal, He is an error signal value output by the fever detection module, Ce is an error signal value output by the cold detection module; Be is an error signal value output by the heart blood pressure detection module, and Se is an error signal value output by the sleep quality detection module, wherein the error signal value represents the absence of an error when being 0, and the discovery of an error when being 1;

if Le=12, then it can be determined that a body temperature sensor is faulted, if Le=15, then it can be determined that a heart rate sensor is faulted, if Le=3, then it can be determined that a blood pressure sensor is faulted, if Le=5, then it can be determined that a blood oxygen sensor is faulted, and if Le is another value, then it can be determined that at least two sensors are faulted.

Preferably, the resource optimization sub-system may include:

a physiological model training module configured to generate a personalized physiological model for the user using an SVM model training method based on radial basis kernel function according to the historical physiological data in the physiological database, store the generated physiological model in the physiological model library, optimize parameters of the physiological model using a cross validation method and periodically update each physiological model in the physiological model library using the SVM model training method based on radial basis kernel function according to the newly collected physiological data; and

a historical data recovery module configured to perform regression fitting processing on the physiological data stored in the physiological database using an SVM model and periodically check whether or not there are physiological data lost and fill a vacancy to repair outliers.

Preferably, the physiological model training module may be configured to: perform the regression fitting processing on physiological data by taking the physiological data collected from the user in the latest period of time and stored in the physiological database as a model training set, generate a personalized physiological model for the user using the SVM model training method based on radial basis kernel function, store the physiological model in the physiological model library and optimize parameters of the physiological model using a cross validation method, wherein a plurality of kinds of dedicated physiological models are stored in the physiological model library for each user aiming at a plurality of kinds of diseases; and

the historical data recovery module may be configured to perform, by taking all historical physiological data of the user as a model training set, the regression fitting processing on physiological data using an SVM model which takes time as an independent variable according to the temporal continuity and stability of the physiological data, output a regression fitting curve of the historical physiological data of the user, perform smoothing processing on outliers according to the regression fitting curve and remedy lost data.

Preferably, the comprehensive evaluation subsystem may include:

a physical sign trend prediction module configured to predict the changing trend and the dynamic change range of the physical signs of the user in the next stage according to the physiological data in the physiological database and the physiological model in the physiological model library using an SVM and a fuzzy information granulation method; and

a comprehensive health evaluation module configured to evaluate the health condition of the user according to the physiological data in the physiological database and the changing trend and the dynamic change range of the physical signs of the user in the next stage using the International detection and evaluation rating scale.

Preferably, the physical sign trend prediction module may be configured to set a fuzzy granularity parameter, perform fuzzy granulation processing on the physiological data stored in the physiological database using triangular fuzzy granules according to the fuzzy granularity parameter, input an SVM for a prediction to obtain an upper limit, a lower limit and a mean level of the next information granule and determine the changing trend and the dynamic change range of the physical signs of the user in the next stage using the three parameters, wherein a relatively small fuzzy granularity parameter is capable of reflecting a tiny change of the body of the user, and a relatively large fuzzy granularity parameter is capable of reflecting the overall changing trend of the user, moreover, the larger the granularity is, the longer the predicable time is.

The disclosure has the following beneficial effects:

The remote home health care system provided herein addresses the problem that existing remote home medical heath care systems are commonly high in false alarm rate and lack of an intelligent personalized health diagnostic technique and commonly causes error historical data and the loss of historical data, realizes an intelligent personalized real-time disease detection and the recovery and maintenance of historically collected data and the health records of a user, provides a reliable health prediction and evaluation strategy and offers the user a reliable real-time pre-dialogists service to help the user know health condition in real time, moreover, the remote home health care system is also capable of discovering certain precursors of a disease or a transient diseases through a long-term monitoring operation to prompt the user to pay more attention or seek for a timely treatment.

The above description is only an overview of the technical solution of the disclosure, to make the technological means of the disclosure better understood and implemented in accordance with the description and the foregoing and other objects, features and advantages of the disclosure readily apparent, specific embodiments of the disclosure are illustrated below.

### BRIEF DESCRIPTION OF THE DRAWINGS

The disclosure will be better understood with reference to the following detailed description of preferred embodiments. The drawings in which identical reference signs denote identical components merely serve to illustrate preferred embodiments of the disclosure but no to limit the disclosure. In the drawings:

FIG. 1 is a structure diagram of a remote home health care system according to an embodiment of the disclosure;

FIG. 2 is a detailed structure diagram of a remote home health care system according to an embodiment of the disclosure;

FIG. 3 is a structure diagram of a fusion sorting subsystem according to an embodiment of the disclosure;

FIG. 4 is a flowchart illustrating health detection and evaluation processing carried out by a remote home health care system according to an embodiment of the disclosure;

FIG. 5 is an internal logic diagram of health detection sub-modules according to an embodiment of the disclosure;

FIG. 6 is a flowchart illustrating a process of establishing a physiological model according to an embodiment of the disclosure; and

FIG. 7 is a flowchart illustrating a process of predicting the changing trend of physical signs according to an embodiment of the disclosure.

### DETAILED DESCRIPTION

Exemplary embodiments of the disclosure are described below with reference to the drawings. Although exemplary embodiments are shown in the drawings, it should be appreciated that the disclosure can be implemented in various forms but not limited to the embodiments set forth here. Contrarily, the embodiments are provided for the thorough understanding of the disclosure and for the complete conveyance of the scope of the disclosure to those skilled in the art.

To address the problem that existing remote home medical health care systems are commonly high in false alarm rate and lack of an intelligent personalized health diagnostic technique and commonly causes error historical data and the loss of historical data, the disclosure provides a remote home health care system which is described below in detail with reference to embodiments when read conjunction with the drawings. It should be appreciated that the specific embodiments described here are merely illustrative of the disclosure but not to be construed as limiting the disclosure.

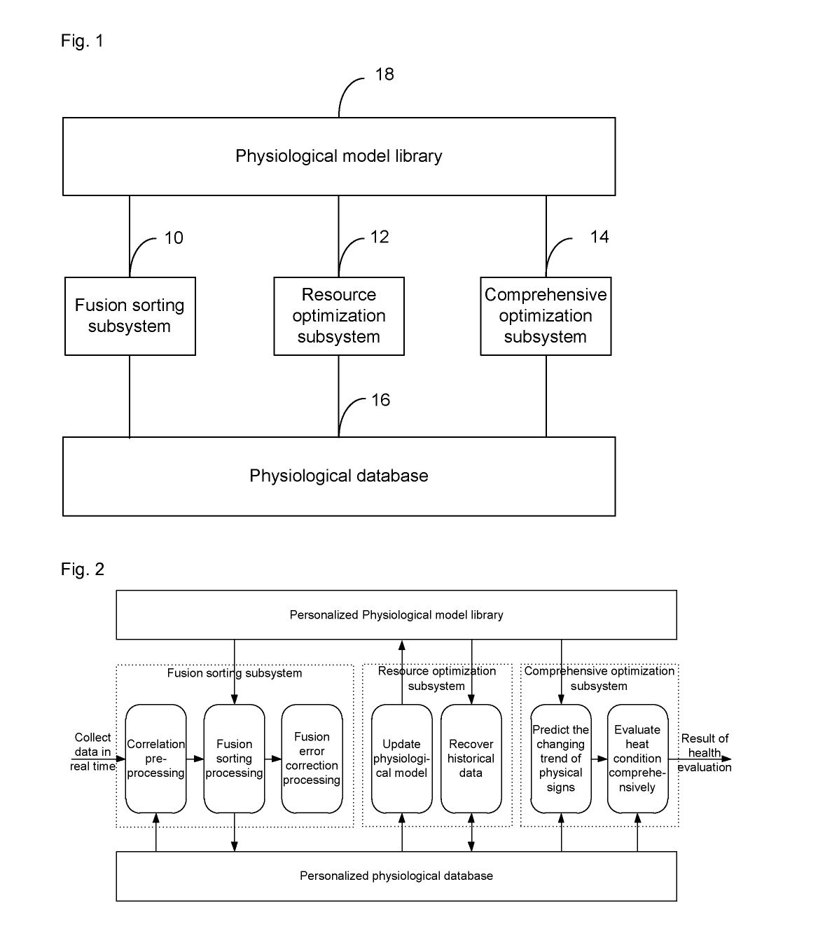

In accordance with an embodiment of the disclosure, a remote home health care system is provided, FIG. 1 is a structure diagram of a remote home health care system according to an embodiment of the disclosure, as shown in FIG. 1, a remote home health care system according to an embodiment of the disclosure includes: a fusion sorting subsystem 10, a resource optimization subsystem 12, a comprehensive optimization subsystem 14, a physiological database 16 and a physiological model library 18, each of which is described below in detail.

The fusion sorting subsystem 10 is configured to receive physical sign data parameters collected by a sensor in real time, perform fusion sorting processing on the physical sign data parameters, pre-diagnose the physical condition of a user in real time according to the physical sign data parameters and a physiological model in the physiological model library 18 while discovering and filtering error data in the physical sign data parameters, and store the data resulting from the fusion sorting processing and the physical sign data parameters in the physiological database 16 as physiological data.

Preferably, in an embodiment of the disclosure, the physiological data further includes: electronic medical record, health record and various data needed for the processing of the remote home health care system.

The fusion sorting subsystem 10 includes:

a motion state detection module 106, configured to detect whether the user falls or is in motion according to the physiological data collected by the sensor in real time, give a fall alarm or an abnormal body position alarm and send the fall alarm or the abnormal body position alarm to an alarming module after detecting that the user falls, or send motion information to a health detection module after detecting that the user is in motion;

a health detection module, configured to perform data fusion correlation processing and historical data correlation processing according to the acquired physiological data and motion information, diagnose a disease and discover a physiological data error according to corresponding physiological data and a corresponding physiological model, output a corresponding disease pre-diagnosis result, give a disease alarm if the disease pre-diagnosis result is abnormal, send the disease pre-diagnosis result and the disease alarm to the alarming module, and send a physiological data error signal to an error location module,

wherein the health detection module is further configured to perform the data fusion correlation processing on various acquired physiological data, and in the embodiment, certain medically authoritative formulas may be used during the data fusion correlation processing. The historical data correlation processing is carried out according to the various physiological data collected by the sensor in real time and historical physiological data stored in the physiological database using the following formula 1:

PD(tn)=CP(tn)−NP(tn) Formula 1;

where to is any time of a day, PD is a physical sign difference value, CP is a current value of a certain physical sign, and NP is a physical sign reference value.

Preferably, the health detection module includes: a fever detection module 101 configured to carry out historical data correlation processing according to the various physiological data collected by the sensor in real time and the historical physiological data stored in the physiological database 16, determine whether or not the user has a fever according to the motion information and a corresponding physiological model, discover a physiological data error, output a fever pre-diagnosis result and give a fever alarm if the fever pre-diagnosis result is abnormal, wherein the acquired physiological data includes: a body temperature parameter and a heart rate parameter; a cold detection module 102 configured to carry out historical data correlation processing according to the various physiological data collected by the sensor in real time and the historical physiological data stored in the physiological database 16, determine whether or not the user has a cold according to the motion information, corresponding physiological data and a corresponding physiological model, discover a physiological data error, output a cold pre-diagnosis result and give a fever alarm if the cold pre-diagnosis result is abnormal, wherein the acquired physiological data includes: a body temperature parameter, a heart rate parameter and a blood oxygen parameter; a heart blood pressure detection module 103 configured to carry out data fusion correlation processing using a medically authoritative formula according to the heart rate parameter, a systolic pressure parameter and a diastolic pressure parameter contained in the various physiological data collected by the sensor in real time, perform historical data correlation processing on original input parameters, the parameters resulting from the fusion processing, that is, an ambulatory pulse pressure, a mean arterial pressure and an ambulatory rate-pressure product, and the historical physiological data stored in the physiological database 16, determine whether or not the user suffers a cardiac disorder and/or a blood pressure disorder according to the motion information, corresponding physiological data and a corresponding physiological model, discover a physiological data error, output a heart blood pressure pre-diagnosis result and give a heat blood pressure alarm if the heart blood pressure pre-diagnosis result is abnormal, wherein the acquired physiological data includes: the heart rate parameter, the systolic pressure parameter and the diastolic pressure parameter; and a sleep quality detection module 104 configured to carry out data fusion correlation processing using a medically authoritative formula according to the heart rate parameter, the systolic pressure parameter and the diastolic pressure parameter contained in the various physiological data collected by the sensor in real time, perform historical data correlation processing on original input parameters, the parameters resulting from the fusion processing, that is, the ambulatory pulse pressure, the mean arterial pressure and the ambulatory rate-pressure product, and the historical physiological data stored in the physiological database 16, determine whether or not the sleep quality of the user is abnormal according to the motion information, corresponding physiological data and a corresponding physiological model, discover a physiological data error, output a sleep quality pre-diagnosis result and give a sleep quality alarm if the sleep quality pre-diagnosis result is abnormal, wherein the acquired physiological data includes: the heart rate parameter, the systolic pressure parameter, the diastolic pressure parameter and the blood oxygen parameter; and

an error location module 105 configured to: locate a faulted sensor according to a physiological data error signal sent by the health detection module and start a sensor error alarm to prompt the user to check the corresponding sensor.

The error location module 105 is further configured to: start a re-transmission mechanism for the faulted sensor after locating the faulted sensor, start a sensor error alarm to prompt the user to check the corresponding sensor if the error still exists after re-transmission is carried out more than a predetermined threshold times.

Preferably, the error location module 105 is further configured to acquire a location output signal according to the following formula 2:

Le=He*23+Ce*22+Be*21+Se*20 Formula 2;

where Le is a location output signal, He is an error signal value output by the fever detection module 101, Ce is an error signal value output by the cold detection module 102; Be is an error signal value output by the heart blood pressure detection module 103, Se is an error signal value output by the sleep quality detection module 104, wherein the error signal value represents the absence of an error when being 0 and the discovery of an error when being 1; if Le=12, then it can be determined that a body temperature sensor is faulted, if Le=15, then it can be determined that a heart rate sensor is faulted, if Le=3, then it can be determined that a blood pressure sensor is faulted, if Le=5, then it can be determined that a blood oxygen sensor is faulted, and if Le is another value, then it can be determined that at least two sensors are faulted.

The alarming module is configured to conduct a comprehensive calculation according to the fall alarm or the abnormal position body alarm sent from the motion state detection module 106 and the disease pre-diagnosis result and the disease alarm sent from the health detection module, output final alarm information, automatically give an alarm to a medical institution and/or a family member of the user after determining that the user is in danger according to the final alarm information, and send the currently abnormal physiological data of the user.

The resource optimization sub-system 12 is configured to optimize the physiological data in the physiological database 16 periodically, generate a personalized physiological model for a user according to the historical physiological data in the physiological database 16, store the generated physiological model in the physiological model library 18, and update the physiological model in the physiological model library 18 according to the latest physiological data in the physiological database 16;

The resource optimization sub-system 12 includes: a physiological model training module configured to generate a personalized physiological model for a user using an SVM model training method based on radial basis kernel function according to the historical physiological data in the physiological database 16 and store the generated physiological model in the physiological model library 18, optimize parameters of the physiological model using a cross validation method and periodically update each physiological model in the physiological model library 18 using the SVM model training method based on radial basis kernel function according to the newly collected physiological data; and a historical data recovery module configured to perform regression fitting processing on the physiological data stored in the physiological database 16 using an SVM model and periodically check whether or not there are physiological data lost and fill a vacancy to repair outliers.

Preferably, the physiological model training module is configured to perform regression fitting processing on physiological data by taking the physiological data collected from the user in the latest period of time and stored in the physiological database as a model training set, generate a personalized physiological model for the user using the SVM model training method based on radial basis kernel function, store the physiological model in the physiological model library 18 and optimize parameters of the physiological model using a cross validation method, wherein a plurality of kinds of dedicated physiological models are stored in the physiological model library 18 for each user aiming at a plurality of kinds of diseases; and the historical data recovery module is configured to perform, by taking all historical physiological data of the user as a model training set, regression fitting processing on physiological data using an SVM model which takes time as an independent variable according to the temporal continuity and stability of physiological data of the model, output a regression fitting curve of the historical physiological data of the user, perform smoothing processing on outliers according to the regression fitting curve, and remedy lost data.

The comprehensive evaluation subsystem 14 is configured to predict a changing trend and a dynamic change range of the physical signs of the user according to the physiological data in the physiological database 16 and the physiological model in the physiological model library 18, and evaluate the health condition of the user according to the physiological data and a result of the prediction, wherein the physiological database 16 is configured to store the physiological data of the user; and the physiological model library 18 is configured to store the physiological model of the user.

The comprehensive evaluation subsystem 14 includes: a physical sign trend prediction module configured to predict the changing trend and the dynamic change range of the physical signs of the user in a next stage according to the physiological data in the physiological database 16 and the physiological model in the physiological model library 18 using an SVM and a fuzzy information granulation method; and a comprehensive health evaluation module configured to evaluate the health condition of the user according to the physiological data in the physiological database and the changing trend and the dynamic change range of the physical signs of the user in the next stage using the Internal detection and evaluation rating scale.

Preferably, the physical sign trend prediction module is configured to: set a fuzzy granularity parameter, perform fuzzy granulation processing on the physiological data stored in the physiological database using triangular fuzzy granules according to a fuzzy granularity parameter, input an SVM for a prediction to obtain an upper limit, a lower limit and a mean level of the next information granule, and determine the changing trend and the dynamic change range of the physical signs of the user in the next stage using the three parameters, wherein, the fuzzy granularity parameter may be adjusted as required; a relatively small fuzzy granularity parameter is capable of reflecting a tiny change in the body of the user, and a relatively large fuzzy granularity parameter is capable of reflecting the overall changing trend of the user, moreover, the larger the granularity is, the longer the predicable time is.

The foregoing technical solutions of the disclosure are described below in detail with reference to the drawings.

FIG. 2 is a detailed structure diagram of a remote home health care system according to an embodiment of the disclosure, as shown in FIG. 2, in an embodiment of the disclosure, a remote home health care system which may be constructed in a background server of a remote home medical monitoring system includes three subsystems of a fusion sorting subsystem, a resource optimization subsystem and a comprehensive evaluation subsystem, a personalized physiological database and a model library. The fusion sorting subsystem needs to carry out correlation pre-processing, fusion sorting processing and fusion error correction processing; the resource optimization subsystem includes two processing modules of a physiological model training module and a historical data recovery module; the comprehensive evaluation subsystem includes a physical sign trend prediction module and a comprehensive health evaluation module; the personalized physiological database is used to store physical sign data, electronic medical records and health records collected from a user and various data needed for various processing; and the personalized physiological model library in which each physiological model of each user is stored is an important intelligent diagnostic tool.

In the remote home health care system provided herein, the fusion sorting subsystem takes charge of receiving the physical sign data collected in real time, carrying out a series of fusion and sorting processing, pre-diagnosing the physical condition of the user and feeding back the result of the pre-diagnosis in real time while discovering and filtering an error signal before the data is sent to a database so as to obtain relatively pure physical sign data; the resource optimization sub-system periodically checks the historical data in the database to determine whether or not there is historical data lost, fills a vacancy, repairs relatively large outliers and periodically updates the personalized physiological model using newly collected data; and the comprehensive evaluation subsystem predicts a changing trend and a dynamic change range of the physical signs in a next stage using historically collected user data and evaluates the health condition of the user from a plurality of aspects according to a questionnaire answered by the user in combination with the electronic medical records and the health records of the user.

The foregoing subsystems are described below in detail.

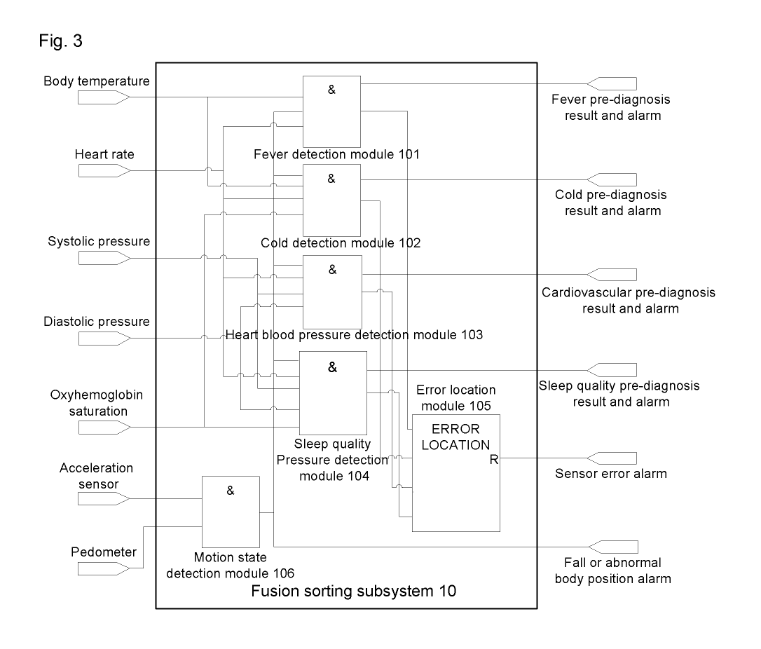

FIG. 3 is a structure diagram of the fusion sorting subsystem according to an embodiment of the disclosure, as shown in FIG. 3, the fusion sorting subsystem receives a plurality of physical sign parameters collected from a user in real time, first, the motion state detection module 106 detects whether or not the user falls accidentally and whether or not the user is in motion, and sends motion information to each health detection sub-module. The fever detection module 101, the cold detection module 102, the heart blood pressure detection module 103 and the sleep quality detection module 104 separately select needed relative input, sequentially perform a data fusion correlation processing and a historical data correlation processing, and then judge the condition of the user and discover an error using a personalized SVM fusion sorting module.

The error location module 105 receives error signals from the fever detection module, the cold detection module, the heart blood pressure detection module and the sleep quality detection module, locates a faulted sensor, that is, determines which sensor is failed, through logical reasoning, calculation and decoding, starts a re-transmission mechanism for the faulted sensor, and starts a sensor error alarm to prompt the user to check the sensor if the error still exists after conducting re-transmission twice. At last, the alarming module outputs a feedback and alarm information according to the detection results of the health detection sub-module and the motion state detection sub-module and the output result of the error location module 105. In other words, the alarming module conducts a comprehensive calculation according to a fall alarm or an abnormal position body alarm sent from the motion state detection module 106, the motion information and the disease pre-diagnosis result and the disease alarm sent from the health detection module, outputs final alarm information, automatically gives an alarm to a medical institution and/or a family member of the user after determining that the user is in danger according to the final alarm information and sends the currently abnormal physiological data of the user.

Resource Optimization Subsystem

The resource optimization subsystem includes a physiological model training module and a historical data recovery module. The physiological model training module periodically updates each physiological model in the personalized physiological model library according to the newly collected user data using an SVM model training method based on radial basis kernel function so that the physiological models can timely follow up the change in the physical condition of the user. The historical data recovery module performs regression fitting processing on the user historical data stored in the database using an SVM model, and periodically checks whether or not there are physiological data lost and fill a vacancy to repair outliers, thus guaranteeing the integrity and the accuracy of collected records and health records.

Comprehensive Evaluation Subsystem

The comprehensive evaluation subsystem includes a physical sign trend prediction part and a comprehensive health evaluation part. The comprehensive evaluation subsystem combines a support vector machine with a fuzzy information granulation method to predict the changing trend and the dynamic change range of the physical signs of the user in the next stage based on historically collected data. Then, the health condition of the user is evaluated from a plurality of aspects using the International detection and evaluation rating scale in combination with a questionnaire answered by the user and the electronic medical records and the health records of the user. At last, a corresponding health service is provided according to the result of the evaluation.

Personalized Medical Database and Model Library

The personalized medical database is configured to store the physical sign data, the electronic medical records and the health records collected from the user and various data needed for various processing. The user historical data stored in the personalized medical database is first processed by the fusion sorting subsystem to filter error information, then the error data or the data lost is repaired by the resource optimization subsystem, thereby guaranteeing the integrity and the validity of the historical data. The data is configured to train a personalized physiological model and predict the changing trend of physical signs and also serves as a good data resource for health evaluation.

The personalized physiological model library for storing each physiological model of each user is an important intelligent diagnostic tool. The personalized physiological model libraries formed by training the great amount of historical physiological data of each user are stored in a personalized medical model library. As the fusion sorting of information is real-time, not allowing a model to be trained in real time, it is necessary to call a trained model. Usually, the physiological model library is updated weekly or every several days, needing no real-time update, however, in a case where the health condition of the user changes drastically, it is needed to update the physiological model library in real time.

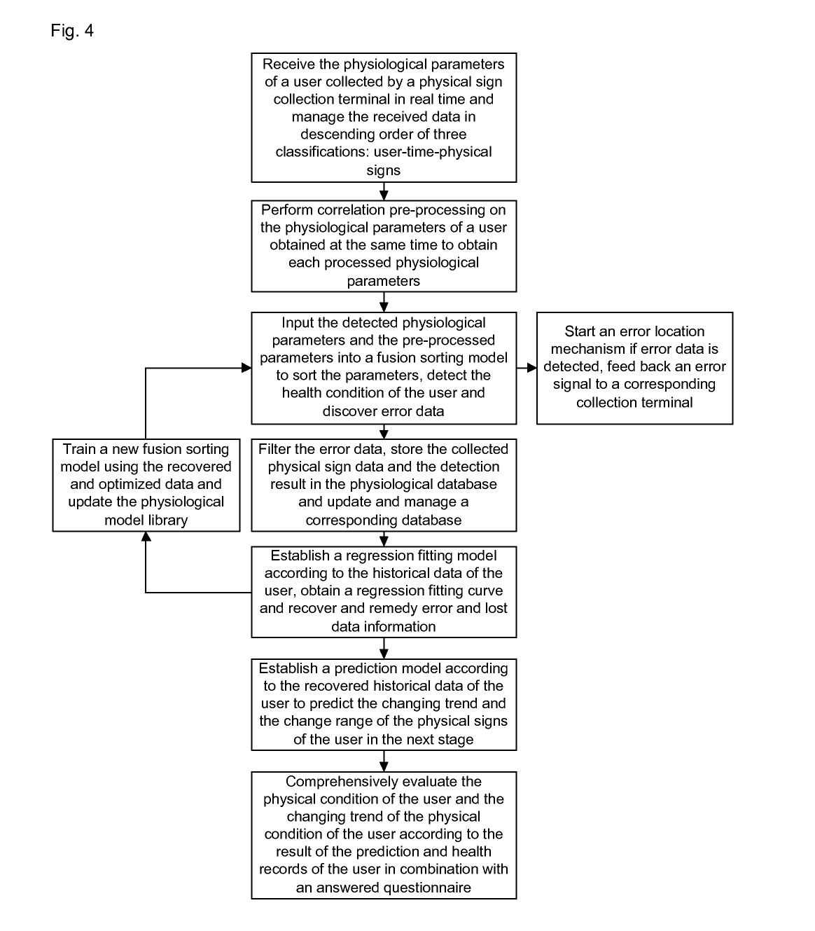

FIG. 4 is a flowchart illustrating health detection and evaluation processing carried out by a remote home health care system according to an embodiment of the disclosure, as shown in FIG. 4, the health detection and evaluation processing includes the following steps:

Step 1: the fusion sorting subsystem receives the physiological parameters of a user uploaded by a physical sign collection terminal in real time and manages the received data in descending order of three classifications: user-time-physical signs;

Step 2: as shown in FIG. 3, first, the motion state detection module 106 detects whether or not the user falls accidentally and whether or not the user is in a motion state, and sends motion information (mainly a step number) to each health detection sub-module;

Step 3: the fever detection module 101, the cold detection module 102, the heart blood pressure detection module 103 and the sleep quality detection module 104 separately select needed relative input, the fever detection sub-module inputs a body temperature parameter and a heart rate parameter; the cold detection sub-module inputs the body temperature parameter, the heart rate parameter and a blood oxygen parameter; the heart blood pressure detection sub-module inputs the heart rate parameter, a systolic pressure parameter and a diastolic pressure parameter; and the sleep quality detection module 104 inputs heart rate parameter, a blood pressure parameter and the blood oxygen parameter; meanwhile, each health detection sub-module inputs step number information;

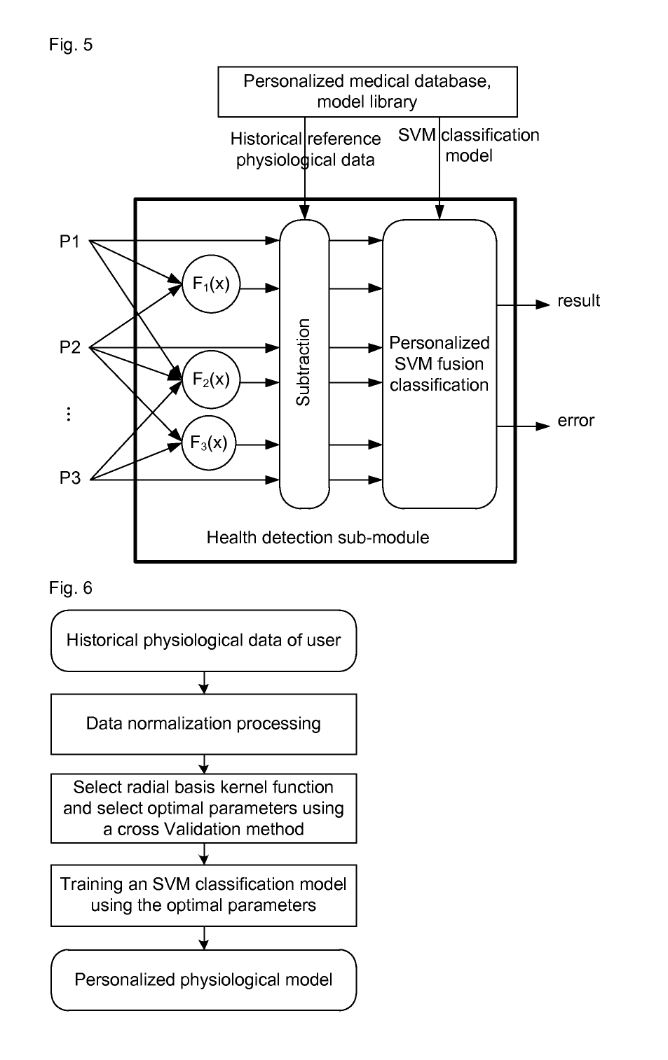

Step 4: the internal logic of each health detection sub-module is shown in FIG. 5, to provide a more accurate and effective pre-diagnosis result, each sub-module first performs certain correlation processing on input signals. In an embodiment of the disclosure, signal correlation processing is generally implemented through two steps: correlation processing based on data fusion and correlation processing based on historical data. When implemented in different sub-modules, the two processing have different implementation steps, wherein the heart blood pressure detection module 103 and the sleep quality detection module 104 carry out both the data correlation processing based on data fusion and the historical data correlation processing sequentially, the fever detection module 101 and the cold detection module 102 merely carry out a historical data correlation processing, and the motion state detection module 106 carries out no correlation processing.

Correlation Processing Based on Data Fusion

In the heart blood pressure diagnosis module and the sleep quality diagnosis module, input signals include Heart Rate (HR), Systolic Pressure (SP) and Diastolic Pressure (DP). It is medically known that parameters ‘Ambulatory Pulse Pressure’ (APP), Mean Arterial Pressure (MAP) and Ambulatory Rate-Pressure Product (ARPP) are more effective in the diagnosis of cardiovascular diseases. Therefore, the input signals are fused according to the following three medically authoritative formulas, and then the original input parameters and the fused parameters APP, MAP and ARPP are sent backwards together.

APP=F1(SP,DP)=SP−DP

MAP=F3(SP,DP)=DP+(SP−DP)/3

ARPP=F2(HR,SP)=HR*SP

Correlation Processing Based on Historical Data

The four health detection sub-modules all need to carry out the correlation processing based on historical data for the sake that the physical sign parameters of a human body change slightly within a day, and it is likely to give an error diagnosis if the change of the physical signs is not taken into consideration. Thus, further correlation processing based on historical data needs to be implemented. The historical data are daily physical sign values which are detected from a user in normal condition as Normal Parameters (NP) and stored in the personal physiological database of the use. A Parameter Difference (PD) is obtained by subtracting a Current Parameter (CP) from the reference value of the parameter obtained at the same moment. PD(tn)=CP(tn)−NP(tn), where to represents any time of a day, apparently, PD is more effective in classification than CP, and classification precision can be greatly improved with the use of PD as the input of a classifier.

Step 5: each health detection sub-module carries out a series of correlation processing on input parameters, pre-diagnoses a disease and discovers an error using a personalized SVM fusion classification model. The fusion model for each disease is trained and periodically updated by the resource optimization subsystem and stored in a personalized physiological model library.

Different pre-diagnosis results may be obtained by judging the classification of different physical sign parameters using each fusion model, and health conditions that can be pre-diagnosed include: ‘normal’, ‘several recognizable abnormalities’ and ‘error information found’. For example, in the heart blood pressure detection module 103, the results output by a fusion model includes: ‘normal’, ‘hypertension’, ‘hypotension’ and ‘error’. Signals representing normal and abnormality are output from a result port, and a signal representing an error is output from an error port.

Step 6: the error location module 105 receives error signals sent from the fever detection module, the cold detection signal, the heart blood pressure detection module and the sleep quality detection module, locates a faulted sensor, that is, determines which sensor is failed, through logical reasoning, calculation and decoding, starts a re-transmission mechanism for the faulted sensor, and starts a sensor error alarm to prompt the user to check the sensor if the error still exists after conducting re-transmission twice.

An error signal location method is as follows: it is assumed that in the error signals output from each fusion detection sub-module, 1 represents an error and 0 represents no error. Error signals output from the fever detection module, the cold detection signal, the heart blood pressure detection module and the sleep quality detection module are represented by He, Ce, Be and Se, respectively, and a location output signal is represented by Le, then Le=He*23+Ce*22+Be*21+SE*20, Le being 12 indicates that a body temperature sensor is faulted, Le being 15 indicates that a heart rate sensor is faulted, Le being 3 indicates that a blood pressure sensor is faulted, Le being 5 indicates that a blood oxygen sensor is faulted, and Le being others indicates an error location or that more than one sensor is faulted.

Step 7: alarm information is output according to detection results of the health detection sub-module and the motion state detection sub-module and the output result of the error location module 105. If an error location signal is received, then a re-transmission mechanism is started regardless of detection results of the other modules, and a sensor error alarm is started if the re-transmission is ineffective. When an emergency situation is detected, a gateway automatically gives an alarm to the nearest medical institution or a family member of the patient, and the basic information of the patient, the current physical sign parameters and the current condition of the patient are sent to the monitoring personnel in the hospital through a network.

Step 8: each physiological model in the personalized physiological model library is periodically updated according to the newly collected data so that the physiological models can timely follow up the changing trend of the physical condition of the user. In the physiological model establishment method shown in FIG. 6, the physiological data that are collected from a user in the past weeks or months and stored in the database are used as a model training set. As different physiological parameters are not in the same physical dimension, it is needed to perform normalization preprocessing on data, that is, unify the original data into a range [0, 1], before the data is trained. To get an ideal classification, model parameters are optimized using a cross validation method and an SVM classification model which uses a radial basis kernel function as a kernel function. Then, a support vector machine is trained, the model resulting from the training can replace the formerly trained model, thus periodically updating the model library. The fusion model is formed by training the great amount of historical physiological data of each user to meet the requirement on personalized diagnosis. Moreover, each disease has a corresponding SVM fusion model, that is, each user has a plurality of dedicated fusion models. The fusion sorting subsystem can complete a real-time detection and classification merely by calling a needed model when performing fusion processing on the data collected.

Step 9: in the regression fitting of historical data, the physiological records collected from the user or even all historical data of the user is used as a model training set. Based on the temporal continuity and stability of physical sign data, regression fitting processing is performed on the historical physiological data of the user using an SVM model which takes time as an independent variable, and at last, the regression fitting curve of the historical data of a certain physical sign of the user is output. The result of the regression fitting is basically matched with original values except that a few outliers are smoothed and several lost data are remedied. The physiological database needs to be repaired periodically so as to guarantee the accuracy and the effectiveness of physiological model training data and the integrity and the reliability of health evaluation data information.

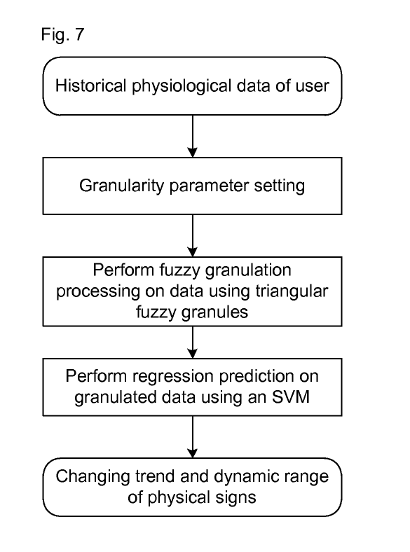

Step 10: the changing trend and the dynamic change range of the physical signs of the user in the next stage are predicted using the historical data of the user. As shown in FIG. 7, a physical sign trend prediction method combines an SVM with a fuzzy information granularity method to effectively predict the changing trend and the change range of physiological parameters of a human body. First, a fuzzy granularity parameter is set, a small granularity is capable of reflecting a tiny physical change of the user while a large granularity is capable of reflecting the overall changing trend of physical signs of the user, moreover, the larger the granularity is, the longer the predictable time is, thus, the granularity parameter should be properly large in a prediction model but should not be too large, otherwise, the predicted dynamic range is too wide to have a prediction effect. Then, a fuzzy granulation processing is performed on data using triangular fuzzy granules to obtain the upper limit, the lower limit and the mean level of each granule which may be represented by three parameters: up, low and r. The subsystems performs a fuzzy information granulation processing on the historical data stored in the personalized physiological database and then inputs an SVM to predict the parameters ‘up’, ‘low’ and ‘r’ of the next information granule. The changing trend and the dynamic change range of the physiological data in the next stage can be obtained based on the three parameters. The changing trend of physical signs is predicted based on complete and effective historical physiological data and the support of an SVM physiological model, which both rely on the assistance of the fusion sorting subsystem and the resource optimization subsystem.

Step 11: the health condition of the user is predicted from a plurality of aspects according to a questionnaire answered by the user in combination with the electronic medical records and the health records of the user. A comprehensive health evaluation may be carried out according to each predicted physical sign parameter, the health records and the medical records of the user in combination with the International detection and evaluation rating scale. With the use of a questionnaire, evaluated contents can be extended in many aspects, including living quality, dietary habit, social environment, psychological health and sub-health level, and a corresponding health evaluation value can be obtained using an option scoring mechanism and a weighting method. At last, a corresponding health service is provided according to the evaluation result.

In conclusion, by means of the technical solution provided herein, the remote home health care system provided herein addresses the problem that existing remote home medical heath care systems are commonly high in false alarm rate and lack of an intelligent personalized health diagnostic technique and commonly causes error historical data and the loss of historical data, realizes an intelligent personalized real-time disease detection and the recovery and maintenance of historically collected data and the health records of a user, provides a reliable health prediction and evaluation strategy, offers the user a reliable real-time pre-dialogists service to help the user know health condition in real time, moreover, the remote home health care system is also capable of discovering certain precursors of a disease or a transient diseases through a long-term monitoring operation to prompt the user to pay more attention or seek for a timely treatment.

The algorithms and operations presented herein are not inherently related to any particular computer or other apparatus. Various general-purpose systems may also be used in accordance with the teachings herein. The required structure for a variety of these systems will be apparent to those of skill in the art. In addition, the disclosure is not described with reference to any particular programming language. It should be appreciated that a variety of programming languages may be used to implement the teachings of the disclosure as described herein, and any references to specific languages are provided for disclosure of best implementation mode of the disclosure.

In the description provided herein, numerous specific details are set forth. However, it is understood that embodiments of the disclosure may be practiced without these specific details. In other instances, well-known methods, structures and techniques have not been shown in detail in order not to obscure an understanding of this description.

Similarly it should be appreciated that in the description of exemplary embodiments of the disclosure, various features of the disclosure are sometimes grouped together in a single embodiment, figure, or description thereof for the purpose of simplifying the disclosure and aiding in the understanding of one or more of the various inventive aspects. This method of disclosure, however, is not to be interpreted as reflecting an intention that the claimed disclosure requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this disclosure.

It should be understood by those skilled in the art that the modules in the devices disclosed herein may be adaptively modified and arranged in one or more devices different from embodiments of the disclosure. The modules, units or components included in embodiments of the disclosure may be combined into one module, unit or component or divided into a plurality of sub-modules, sub-units or sub-components. All the features disclosed in the description (including appended claims, abstract and the drawings) and all the procedures or units of any method or device disclosed herein, excluding those features and/or procedures or units which are mutually exclusive, can be combined as any combination. Each feature disclosed in the description (including appended claims, abstract and the drawings) can be replaced by an alternative feature providing identical, equivalent or similar intention.

Additionally, it should be understood by those skilled in the art that although certain embodiments described herein have some features of other embodiments but not other features, the combination of the features of different embodiments falls within the scope of the disclosure and forms a different embodiment. For example, in appended claims, any of the embodiments required to be protected can be employed as any combination.

Embodiments of each component of the disclosure can be implemented as hardware, a software module running on one or more processors or a combination thereof. It should be understood by those skilled in the art that some or all components of some or all components in the remote home care system disclosed herein can be realized using a microprocessor or a Digital Signal Processor (DSP) in practice. The disclosure may also be implemented as a device or apparatus program (e.g. computer program and computer program product) for executing part or all of the methods described herein. Such programs for realizing the disclosure may be stored on a computer-readable medium or may be in the form of one or more signals which can be downloaded from the Internet or provided on a carrier signal or provided in other ways.

It should be noted that the foregoing embodiments are merely illustrative of the disclosure but not to be construed as limiting the disclosure and that replacement embodiments can be devised by those skilled in the art without departing from the scope of appended claims in which each reference sign in a bracket is not to be construed as limiting the claims. The terms ‘comprise’ do not exclude the existence of a component or step not presented in claims. The terms ‘one’ and ‘single’, as used herein in front of a component, do not exclude the existence of a plurality of the same components. The disclosure can be realized by means of hardware including a plurality of different components and a properly programmed computer. In claims concerning a unit listing a plurality of devices, several devices may be specifically implemented as one piece of hardware. The terms ‘first’, ‘second’ and ‘third’ which are not used to limit a sequence can be interpreted as names.

### INDUSTRIAL APPLICATION

The remote home health care system provided herein addresses the problem that existing remote home medical heath care systems are commonly high in false alarm rate and lack of an intelligent personalized health diagnostic technique and commonly causes error historical data and the loss of historical data, realizes an intelligent personalized real-time disease detection and the recovery and maintenance of historically collected data and the health records of a user, provides a reliable health prediction and evaluation strategy, offers the user a reliable real-time pre-dialogists service to help the user know health condition in real time, moreover, the remote home health care system is also capable of discovering certain precursors of a disease or a transient diseases through a long-term monitoring operation to prompt the user to pay more attention or seek for a timely treatment.

What is claimed is:

1. A remote home health care system, comprising:
a fusion sorting subsystem configured to receive physical sign data parameters collected by a sensor in real time, perform fusion sorting processing on the physical sign data parameters and pre-diagnose a physical condition of a user in real time according to the physical sign data parameters and a physiological model in a physiological model library while discovering and filtering error data in the physical sign data parameters, and store data resulting from the fusion sorting processing in a physiological database as physiological data;
a resource optimization subsystem configured to recover and optimize the physiological data in the physiological database, generate a personalized physiological model for the user according to historical physiological data in the physiological database, store the generated physiological model in the physiological model library, and update the physiological model in the physiological model library according to the latest physiological data in the physiological database;
a comprehensive evaluation subsystem configured to predict a changing trend and a dynamic change range of the physical signs of the user according to the physiological data in the physiological database and the physiological model in the physiological model library, and evaluate a health condition of the user according to the physiological data and the changing trend and the dynamic change range of the physical signs;
a physiological database configured to store the physiological data of the user; and
a physiological model library configured to store the physiological model of the user.

2. The system according to claim 1, wherein the physiological data in the physiological database includes: physical sign data, electronic medical record and health record.

3. The system according to claim 2, wherein the fusion sorting subsystem is further configured to delete the error data in the physical sign data parameters through the fusion sorting processing before storing the physical sign data parameters in the physiological database.

4. The system according to claim 2, wherein the fusion sorting subsystem comprises:
a motion state detection module configured to detect whether the user falls or is in motion according to the physiological data collected by the sensor in real time, give a fall alarm or an abnormal body position alarm and send the fall alarm or the abnormal body position alarm to an alarming module after detecting that the user falls, or send motion information to a health detection module after detecting that the user is in motion;
a health detection module configured to perform data fusion correlation processing and historical data correlation processing according to the acquired physiological data and motion information, diagnose a disease and discover a physiological data error according to corresponding physiological data and a corresponding physiological model, output a corresponding disease pre-diagnosis result, give a disease alarm if the disease pre-diagnosis result is abnormal, send the disease pre-diagnosis result and the disease alarm to the alarming module, and send a physiological data error signal to an error location module;
an error location module configured to receive the physiological data error signal sent from the health detection module, position a faulted sensor and give a sensor error alarm to prompt the user to check a corresponding sensor; and
an alarming module configured to conduct a comprehensive calculation according to the fall or abnormal position body alarm sent from the motion state detection module and the disease pre-diagnosis result and the disease alarm sent from the health detection module, output final alarm information, automatically give an alarm to a medical institution and/or a family member of the user after determining that the user is in danger according to the final alarm information and send the currently abnormal physiological data of the user.

5. The system according to claim 4, wherein the health detection module is configured to:
perform the data fusion correlation processing on the various physiological data acquired;
perform the historical data correlation processing according to the various physiological data collected by the sensor in real time and the historical physiological data stored in the physiological database using the following formula 1:
PD(tₙ)=CP(tₙ)−NP(tₙ) Formula 1
where tₙ is any time of a day, PD is a physical sign difference, CP is a current detection value of a certain physical sign, and NP is a physical sign reference value.

6. The system according to claim 5, wherein the health detection module comprises:
a fever detection module configured to carry out the historical data correlation processing according to the various physiological data collected by the sensor in real time and the historical physiological data stored in the physiological database, determine whether or not the user has a fever according to the motion information and a corresponding physiological model, discover a physiological data error, output a fever pre-diagnosis result and give a fever alarm if the fever pre-diagnosis result is abnormal, wherein the acquired physiological data includes: a body temperature parameter and a heart rate parameter;
a cold detection module configured to carry out the historical data correlation processing according to the various physiological data collected by the sensor in real time and the historical physiological data stored in the physiological database, determine whether or not the user has a cold according to the motion information, corresponding physiological data and a corresponding physiological model, discover a physiological data error, output a cold pre-diagnosis result and give a fever alarm if the cold pre-diagnosis result is abnormal, wherein the acquired physiological data includes: a body temperature parameter, a heart rate parameter and a blood oxygen parameter;
a heart blood pressure detection module configured to carry out the data fusion correlation processing according to the heart rate parameter, a systolic pressure parameter and a diastolic pressure parameter contained in the various physiological data collected by the sensor in real time, perform the historical data correlation processing on original input parameter, the parameters resulting from the fusion processing, that is, an ambulatory pulse pressure, a mean arterial pressure and an ambulatory rate-pressure product, and the historical physiological data stored in the physiological database, determine whether or not the user suffers a cardiac disorder and/or a blood pressure disorder according to the motion information, corresponding physiological data and a corresponding physiological model, discover a physiological data error, output a heart blood pressure pre-diagnosis result and give a heat blood pressure alarm if the heart blood pressure pre-diagnosis result is abnormal, wherein the acquired physiological data includes: a heart rate parameter, the systolic pressure parameter and the diastolic pressure parameter; and
a sleep quality detection module configured to carry out the data fusion correlation processing according to the heart rate parameter, the systolic pressure parameter and the diastolic pressure parameter contained in the various physiological data collected by the sensor in real time, perform the historical data correlation processing on original input parameters, the parameters resulting from the fusion processing, that is, the ambulatory pulse pressure, the mean arterial pressure and the ambulatory rate-pressure product, and the historical physiological data stored in the physiological database, determine whether or not the sleep quality of the user is abnormal according to the motion information, corresponding physiological data and the corresponding physiological model, discover a physiological data error, output a sleep quality pre-diagnosis result and give a sleep quality alarm if the sleep quality pre-diagnosis result is abnormal, wherein the acquired physiological data includes: the heart rate parameter, the systolic pressure parameter, the diastolic pressure parameter and the blood oxygen parameter.

7. The system according to claim 6, wherein the error location module is configured to: start a re-transmission mechanism for a faulted sensor after locating the faulted sensor, start a sensor error alarm to prompt the user to check a corresponding sensor if the error still exists after re-transmission is carried out more than a predetermined threshold times.

8. The system according to claim 6, wherein the error location module is configured to acquire a location output signal according to the following formula 2:
Le=He*2³+Ce*2²+Be*2¹+Se*2⁰ Formula 2
where Le is a location output signal, He is an error signal value output by the fever detection module, Ce is an error signal value output by the cold detection module; Be is an error signal value output by the heart blood pressure detection module, and Se is an error signal value output by the sleep quality detection module, wherein the error signal value represents the absence of an error when being 0, and the discovery of an error when being 1;
if Le=12, then it can be determined that a body temperature sensor is faulted, if Le=15, then it can be determined that a heart rate sensor is faulted, if Le=3, then it can be determined that a blood pressure sensor is faulted, if Le=5, then it can be determined that a blood oxygen sensor is faulted, and if Le is another value, then it can be determined that at least two sensors are faulted.

9. The system according to claim 2, wherein the resource optimization sub-system comprises:
a physiological model training module configured to generate a personalized physiological model for the user using an SVM model training method based on radial basis kernel function according to the historical physiological data in the physiological database, store the generated physiological model in the physiological model library, optimize parameters of the physiological model using a cross validation method and periodically update each physiological model in the physiological model library using the SVM model training method based on radial basis kernel function according to the newly collected physiological data; and
a historical data recovery module configured to perform regression fitting processing on the physiological data stored in the physiological database using an SVM model and periodically check whether or not there are physiological data lost and fill a vacancy to repair outliers.

10. The system according to claim 9, wherein
the physiological model training module is configured to: perform the regression fitting processing on physiological data by taking the physiological data collected from the user in the latest period of time and stored in the physiological database as a model training set, generate a personalized physiological model for the user using the SVM model training method based on radial basis kernel function, store the physiological model in the physiological model library and optimize parameters of the physiological model using a cross validation method, wherein a plurality of kinds of dedicated physiological models are stored in the physiological model library for each user aiming at a plurality of kinds of diseases; and
the historical data recovery module is configured to perform, by taking all historical physiological data of the user as a model training set, the regression fitting processing on physiological data using an SVM model which takes time as an independent variable according to the temporal continuity and stability of the physiological data, output a regression fitting curve of the historical physiological data of the user, perform smoothing processing on outliers according to the regression fitting curve and remedy lost data.

11. The system according to claim 2, wherein the comprehensive evaluation subsystem comprises:
a physical sign trend prediction module configured to predict the changing trend and the dynamic change range of the physical signs of the user in the next stage according to the physiological data in the physiological database and the physiological model in the physiological model library using an SVM and a fuzzy information granulation method; and
a comprehensive health evaluation module configured to evaluate the health condition of the user according to the physiological data in the physiological database and the changing trend and the dynamic change range of the physical signs of the user in the next stage using the International detection and evaluation rating scale.

12. The system according to claim 11, wherein the physical sign trend prediction module is configured to set a fuzzy granularity parameter, perform fuzzy granulation processing on the physiological data stored in the physiological database using triangular fuzzy granules according to the fuzzy granularity parameter, input an SVM for a prediction to obtain an upper limit, a lower limit and a mean level of the next information granule and determine the changing trend and the dynamic change range of the physical signs of the user in the next stage using the three parameters, wherein a relatively small fuzzy granularity parameter is capable of reflecting a tiny change of the body of the user, and a relatively large fuzzy granularity parameter is capable of reflecting the overall changing trend of the user, moreover, the larger the granularity is, the longer the predicable time is.

13. The system according to claim 3, wherein the fusion sorting subsystem comprises:
a motion state detection module configured to detect whether the user falls or is in motion according to the physiological data collected by the sensor in real time, give a fall alarm or an abnormal body position alarm and send the fall alarm or the abnormal body position alarm to an alarming module after detecting that the user falls, or send motion information to a health detection module after detecting that the user is in motion;
a health detection module configured to perform data fusion correlation processing and historical data correlation processing according to the acquired physiological data and motion information, diagnose a disease and discover a physiological data error according to corresponding physiological data and a corresponding physiological model, output a corresponding disease pre-diagnosis result, give a disease alarm if the disease pre-diagnosis result is abnormal, send the disease pre-diagnosis result and the disease alarm to the alarming module, and send a physiological data error signal to an error location module;
an error location module configured to receive the physiological data error signal sent from the health detection module, position a faulted sensor and give a sensor error alarm to prompt the user to check a corresponding sensor; and
an alarming module configured to conduct a comprehensive calculation according to the fall or abnormal position body alarm sent from the motion state detection module and the disease pre-diagnosis result and the disease alarm sent from the health detection module, output final alarm information, automatically give an alarm to a medical institution and/or a family member of the user after determining that the user is in danger according to the final alarm information and send the currently abnormal physiological data of the user.

14. The system according to claim 13, wherein the health detection module is configured to:
perform the data fusion correlation processing on the various physiological data acquired;
perform the historical data correlation processing according to the various physiological data collected by the sensor in real time and the historical physiological data stored in the physiological database using the following formula 1:
PD(tₙ)=CP(tₙ)−NP(tₙ) Formula 1
where tₙ is any time of a day, PD is a physical sign difference, CP is a current detection value of a certain physical sign, and NP is a physical sign reference value.

15. The system according to claim 14, wherein the health detection module comprises:
a fever detection module configured to carry out the historical data correlation processing according to the various physiological data collected by the sensor in real time and the historical physiological data stored in the physiological database, determine whether or not the user has a fever according to the motion information and a corresponding physiological model, discover a physiological data error, output a fever pre-diagnosis result and give a fever alarm if the fever pre-diagnosis result is abnormal, wherein the acquired physiological data includes: a body temperature parameter and a heart rate parameter;
a cold detection module configured to carry out the historical data correlation processing according to the various physiological data collected by the sensor in real time and the historical physiological data stored in the physiological database, determine whether or not the user has a cold according to the motion information, corresponding physiological data and a corresponding physiological model, discover a physiological data error, output a cold pre-diagnosis result and give a fever alarm if the cold pre-diagnosis result is abnormal, wherein the acquired physiological data includes: a body temperature parameter, a heart rate parameter and a blood oxygen parameter;
a heart blood pressure detection module configured to carry out the data fusion correlation processing according to the heart rate parameter, a systolic pressure parameter and a diastolic pressure parameter contained in the various physiological data collected by the sensor in real time, perform the historical data correlation processing on original input parameter, the parameters resulting from the fusion processing, that is, an ambulatory pulse pressure, a mean arterial pressure and an ambulatory rate-pressure product, and the historical physiological data stored in the physiological database, determine whether or not the user suffers a cardiac disorder and/or a blood pressure disorder according to the motion information, corresponding physiological data and a corresponding physiological model, discover a physiological data error, output a heart blood pressure pre-diagnosis result and give a heat blood pressure alarm if the heart blood pressure pre-diagnosis result is abnormal, wherein the acquired physiological data includes: a heart rate parameter, the systolic pressure parameter and the diastolic pressure parameter; and
a sleep quality detection module configured to carry out the data fusion correlation processing according to the heart rate parameter, the systolic pressure parameter and the diastolic pressure parameter contained in the various physiological data collected by the sensor in real time, perform the historical data correlation processing on original input parameters, the parameters resulting from the fusion processing, that is, the ambulatory pulse pressure, the mean arterial pressure and the ambulatory rate-pressure product, and the historical physiological data stored in the physiological database, determine whether or not the sleep quality of the user is abnormal according to the motion information, corresponding physiological data and the corresponding physiological model, discover a physiological data error, output a sleep quality pre-diagnosis result and give a sleep quality alarm if the sleep quality pre-diagnosis result is abnormal, wherein the acquired physiological data includes: the heart rate parameter, the systolic pressure parameter, the diastolic pressure parameter and the blood oxygen parameter.

16. The system according to claim 15, wherein the error location module is configured to: start a re-transmission mechanism for a faulted sensor after locating the faulted sensor, start a sensor error alarm to prompt the user to check a corresponding sensor if the error still exists after re-transmission is carried out more than a predetermined threshold times.

17. The system according to claim 15, wherein the error location module is configured to acquire a location output signal according to the following formula 2:
Le=He*2³+Ce*2²+Be*2¹+Se*2⁰ Formula 2
where Le is a location output signal, He is an error signal value output by the fever detection module, Ce is an error signal value output by the cold detection module; Be is an error signal value output by the heart blood pressure detection module, and Se is an error signal value output by the sleep quality detection module, wherein the error signal value represents the absence of an error when being 0, and the discovery of an error when being 1;
if Le=12, then it can be determined that a body temperature sensor is faulted, if Le=15, then it can be determined that a heart rate sensor is faulted, if Le=3, then it can be determined that a blood pressure sensor is faulted, if Le=5, then it can be determined that a blood oxygen sensor is faulted, and if Le is another value, then it can be determined that at least two sensors are faulted.
